(19) United States
(12) Patent Application Publication
(10) Pub. No.: US 2009/0285911 A1
(43) Pub. Date: Nov. 19, 2009

(54) Cynara scolymus extracts, the use thereof and formulations containing them

(76) Inventors: Ezio Bombardelli, Groppello Cairoli (IT); Gabriele Fontana, Milano (IT); Andrea Giori, Milano (IT); Paolo Morazzoni, Milano (IT); Massimo Ronchi, Milano (IT); Sabrina Arpini, Milano (IT)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 11/988,638

(22) PCT Filed: Jun. 16, 2006
(86) PCT No.: PCT/EP2006/005778

§ 371 Date: Mar. 10, 2009

(30) Foreign Application Priority Data
Jul. 14, 2005 (IT) MI2005A001347

(51) Int. Cl. A61K 36/28; A61K 31/343; A61P 3/00; A61P 1/00; A61P 9/10

(52) U.S. Cl. 424/725; 514/468

(57) ABSTRACT

The present invention relates to the preparation of a Cynara scolymus extract obtainable by fractioning on a resin. The process of the invention allows to obtain an extract, starting from the aerial parts of the plant Cynara scolymus, containing three classes of active principles, namely dicaffeoylquinic acids, luteolin and cynaropicrin glycosides, in a constant ratio. Cynaropicrin is stabilized by addition of precise amounts of sulfated amino acids or suitable thio-derivatives. These extracts have hypolipemizing, anti-dyspeptic and vascular anti-inflammatory activities. The extracts are mainly formulated in Enothera biennis oil or in oils rich in ω-3 and ω-6 acids which enhance the vascular activity.

The present invention relates to the preparation of an extract of Cynara scolymus thorny varieties obtainable by fractioning on a resin and to the process for its preparation.

The process of the invention allows to obtain an extract, starting from the aerial parts of the thorny varieties of Cynara scolymus, containing three classes of active principles, namely dicaffeoylquinic acids, luteolin and cynaropicrin glycosides, in a constant ratio. Cynaropicrin is stabilized by addition of precise amounts of sulfated amino acids or suitable thio-derivatives. These extracts have hypolipemizing, anti-dyspeptic and vascular anti-inflammatory activities. The extracts are mainly formulated in oils rich in ω-3 and ω-6 acids which enhance the vascular activity.

### TECHNOLOGICAL BACKGROUND

It is known from literature that the aqueous or water-alcohol Cynara scolymus extracts have hypocholesterolemizing, choleretic and anti-dyspeptic activities. The hypocholesterolemizing activity has been known for many years and it concerns two classes of substances: cynarin, a dicaffeoylquinic acid prepared by synthesis and used in therapy until the '70s, and flavonoids, which proved to have in vitro inhibiting activity on hepatic cholesterol synthesis. The global activity is related to the choleretic action, peculiar to Cynara scolymus extracts, which promotes cholesterol removal through the removal of bile acids.

Cynara scolymus active principles are potent antioxidant agents which are easily degraded when drying the vegetable material. The preparation of the vegetable biomass is therefore crucial to obtain extracts with high content in active principles.

Cynaropicrin is a terpene having anti-inflammatory activity and mild hypocholesterolemizing action. As is the case with all methylene-gamma lactonic ring sesquiterpenes, cynaropicrin is poorly stable and this is one of the reasons why this compound is not present in extracts and formulations. On the other hand, the presence of this compound in the extracts is crucial, because bioavailable substances with anti-inflammatory action, that can modulate vascular inflammation (NFkB and reactive protein C) are particularly suitable for the prevention and therapy of arteriosclerosis and heart diseases.

### DISCLOSURE OF THE INVENTION

The present invention relates to novel extracts of Cynara scolymus thorny varieties, preferably Cynara scolymus var. Spinoso sardo or Cynara scolymus var. tema containing three classes of active principles, dicaffeoylquinic acids, luteolin and cynaropicrin glycosides, in constant ratios. The present invention further relates to the process for the preparation of said extract.

It has surprisingly been found that the addition of sulfated amino acids, preferably cysteine, during the extraction step or the subsequent processes for the purification or concentration of the Cynara scolymus extracts, provides final extracts still containing high concentrations of cynaropicrin, which remains stable in the therapeutical formulations. In fact the sulfated amino acids give rise to adducts which stabilize the sesquiterpene and promote its absorption. In the plasma, these adducts undergo exchange reaction with protein sulfhydryl groups thus restoring cynaropicrin specific activity.

An important therapeutical use of the extract of the invention concerns the treatment of irritable colon, a disease affecting up to 9% of western countries population. The suggested anti-inflammatory action related to the modulation of NFkB, TNF-α and some interleukins can be one of the mechanisms involved in the alleviation of irritable colon symptoms.

Sesquiterpen-lactones also interact, either directly or indirectly, with central nervous system mediators involved in intestinal diseases.

According to the invention, when the sulfated amino acids are not used as stabilizing agents during the extraction and/or concentration steps, the extract can be stabilized by addition of sulfated amino acids to the formulations.

The extracts are prepared using the aerial parts of the plant, including capitula which are the richer in dicaffeoylquinic acids part; leaves mainly contain flavonoids and all of the cynaropicrin.

According to the invention, the whole fresh or dehydrated plant, preferably the fresh one, can be used, in fixed ratios between capitula and the remaining aerial part ranging from 20:80 to 40:60, preferably 30:70.

As already mentioned, the preparation of the vegetable biomass is crucial to avoid degradation of active principles when drying the vegetable material.

According to the invention, the vegetable material can be frozen immediately after collection to decrease the action of the many oxidases and hydrolases naturally occurring in the plants. The frozen biomass is ground at −30° C. and immediately immersed in the extraction alcohol solvent, which completes the enzyme inactivation as well as the extraction of the active principles. Water-alcohol solutions are used for extracting all of the active principles, preferably 70% solutions which provide the best ratio among the various components.

During extraction, an amount of cysteine 10% higher than the stoichiometric amount of cynaropicrin is added to the solvent. The resulting water-alcohol extracts are concentrated at low temperatures ranging from 25 to 55° C., preferably at 35° C., under water vacuum. During concentration, poorly water-soluble inert substances, such as chlorophylls and some carotenoids usually present in vegetable materials, precipitate and are removed as they do not show any of the activities exerted by the extract of the invention. The aqueous solution is filtered, the solvent and the undesired substances are removed, the resulting solution is concentrated under vacuum to a volume corresponding to that of the extracted vegetable material, and is subsequently purified on an adsorption resin, such as a polystyrene resin, Amberlite, duolite and XAD1180. The resin is thoroughly washed with water and the desired extract is eluted with 90% ethanol until exhaustion of the resin.

The resulting extract according to the present invention has a novel composition compared with the extracts of the prior art; it particularly has a cynaropicrin content >5%, a ratio of caffeoylquinic acids to cynaropicrin ranging from 1:0.2 to 1:0.8, preferably 1:0.6, and a ratio of luteolin glycosides to cynaropicrin ranging from 20 to 60%, preferably 50%.

The extract of the invention was subjected to biological investigation in a series of pharmacological tests. The extract of the invention induced a reduction in cholesterol and triglycerides of 40 and 35%, respectively, in the ethanol-induced hyperlipidemia test, and a dose-dependent reduction in the edema up to 75% in the carrageenin oedema test.

The choleretic action in the rat confirmed the data reported in literature.

The extract is well-suited for incorporation in pharmaceutical formulations such as tablets, sugar-coated pills, soft- and hard- gelatin capsules and cellulose capsules. The extract will preferably be formulated in oils rich in ω-3 and ω-6 polyunsaturated acids such as Enothera biennis oil or Linum usitatissimum oil (flax oil).

Active dosages in humans range from 50 to 1000 mg daily, according to the severity of the disease to treat.

The invention is described in greater detail in the following examples.

### EXAMPLE 1

### Preparation of the Extract of Cynara scolymus var. Spinosa

4.12 kg of whole artichoke plant (capitula, stem and leaves) are extracted in a percolator with 70% ethanol at 70° C. until exhaustion of the vegetable material. Approx. 50 liters of percolate are collected, then concentrated under reduced pressure to remove ethanol. The resulting aqueous concentrate, about 1.5 kg, is centrifuged to separate insolubles. The resulting clear solution is loaded on a chromatographic column containing XAD1180 resin (about 1400 ml), previously conditioned in water. The column is washed with about 2.8 l of water, the eluate is discarded, and the purified extract is recovered by elution with 3.5 l of 90% v/v ethanol.

The water-alcohol eluate is concentrated, the content in cynaropicrin is checked by HPLC and L-cysteine dissolved in some water is added (in a 10% excess to the stoichiometric of cynaropicrin present). Further concentration and drying at 50° C. under reduced pressure afford 49.4 g of purified dry extract (total caffeoylquinic acids HPLC content 15.1%, total flavonoids HPLC content 3.15%, total cynaropicrin HPLC content 7.64%).

### EXAMPLE 2

### Preparation of the Extract of Cynara scolymus var. tema

4.12 kg of whole artichoke plant (capitula, stem and leaves) are extracted in a percolator with 70% ethanol at 70° C. until complete extraction of the active principles. The cynaropicrin content in the combined extracts is determined, then L-cysteine is added in a 10% excess to the stoichiometric. The water-ethanol extract is concentrated under vacuum at a temperature not above 30° C. The aqueous concentrate is left to stand overnight at 4° C., then the solid residue, mainly consisting of chlorophyll and carotenoids, is decanted off. The solution is filtered, then absorbed on a XAD 1180 resin which is subsequently washed with water until a complete removal of undesired insolubles. The resin is washed with 90% ethanol and the eluate is concentrated to a 10% w/w residue, which is atomized to afford 40 g of an extract containing 15.8% caffeoylquinic acids, 4.2% luteolin glycosides and 8.6% cynaropicrin.

### EXAMPLE 3

### Preparation of the Extract of Cynara scolymus var. Spinoso sardo

4.12 kg of whole artichoke plant (capitula, stem and leaves) are extracted in a percolator with 70% ethanol at 35° C. until complete extraction of the active principles. The combined extracts are concentrated to 5 l and left to stand in refrigerator for 15 hours. The resulting suspension is centrifuged. The clear solution is absorbed on a XAD 1180 resin which is then washed with water to a dry residue (in the water washings) below 0.01%. The resin is washed with 90% ethanol and the eluate is concentrated at a temperature lower than 40° C. under vacuum, to afford 40 g of an extract containing 16.2% caffeoylquinic acids, 4.02% luteolin glycosides and 8.01% cynaropicrin.

### EXAMPLE 4

### Formulation of Extract in Oily Suspension for Soft-Gelatin Capsules

Unit composition:

| | |
|---|---|
| Extract of example 1 | 200 mg |
| White beeswax | 15 mg |
| Soy lecithin | 20 mg |
| Soy oil | 215 mg |

### EXAMPLE 5

### Formulation of Extract in Oily Suspension for Soft-Gelatin Capsules

Unit composition:

| | |
|---|---|
| Extract of example 2 | 200 mg |
| Soy lecithin | 240 mg |
| White beeswax | 6 mg |
| Enothera biennis oil | 215 mg |

### EXAMPLE 6

### Formulation of Extract in Hard-Gelatin Capsules

Unit composition:

| | |
|---|---|
| Extract of example 1 | 200 mg |
| Microcrystalline cellulose | 200 mg |
| Lactose | 90 mg |
| Silicon dioxide | 5 mg |
| Magnesium stearate | 5 mg |

### EXAMPLE 7

### Formulation of Extract in Hard-Gelatin Capsules

Unit composition:

| | |
|---|---|
| Extract of example 3 | 200 mg |
| L-cysteine | 100 mg |
| Microcrystalline cellulose | 150 mg |
| Lactose | 90 mg |
| Silicon dioxide | 5 mg |
| Magnesium stearate | 5 mg |

### EXAMPLE 8

### Formulation of Extract in Oily Suspension for Soft-Gelatin Capsules

Unit composition:

| | |
|---|---|
| Extract of example 3 | 200 mg |
| Soy lecithin | 240 mg |
| White beeswax | 6 mg |
| Flax oil | 215 mg |

1. A process for the preparation of Cynara scolymus thorny varieties extracts, which comprises:
a) extracting the vegetable fresh or dehydrated biomass with an alcohol or water-alcohol solvent;
b) concentrating the water-alcohol extracts from step a) at low temperatures ranging from 25 to 55° C., preferably at 35° C., under water vacuum;
c) filtering off the precipitated poorly water-soluble inert substances;
d) purifying the extract on an adsorption resin column.

2. A process as claimed in claim 1, in which in step a) a ground vegetable biomass frozen at a temperature of −30° C. is used.

3. A process as claimed in claim 2, in which the vegetable biomass has a ratio of capitula to remaining aerial parts ranging from 20:80 to 40:60.

4. A process as claimed in claim 3, in which the vegetable biomass has a ratio of capitula to remaining aerial parts of 30:70.

5. A process as claimed in claim 1, in which the extraction of step a) is carried out with water-alcohol solutions.

6. A process as claimed in claim 5, in which a 70% water-alcohol solution is used.

7. A process as claimed in claim 5, in which a 70% ethanol solution is used.

8. A process as claimed in claim 1, in which the extraction of step a) is carried out at a temperature ranging from 10° C. to 80° C., preferably at 25° C.

9. A process as claimed in claim 1, in which in step b) the solution is concentrated under vacuum to a volume corresponding to that of the extracted vegetable material.

10. A process as claimed in claim 1, in which in step d) chromatographic separation is carried out on resins selected from polystyrene, Amberlite, duolite, XAD1180 resins.

11. A process as claimed in claim 1, in which in the extraction step a) or in the subsequent purification or concentration step b)—step d), sulfated amino acids, preferably cysteine, are added.

12. A process as claimed in claim 11, in which cysteine is added in an amount 10% higher than the stoichiometric amount of cynaropicrin.

13. A process according to claim 1 in which Cynara scolymus var. Spinoso Sardo is used.

14. A process according to claim 1 in which Cynara scolymus var. tema is used.

15. Cynara scolymus extracts with high content in cynaropicrin, obtainable according to the process of claim 1.

16. Extracts as claimed in claim 15, having a cynaropicrin content higher than 5%, ratio of caffeoylquinic acids to cynaropicrin ranging from 1:0.2 to 1:0.8, preferably 1:0.6, and luteolin glycosides to cynaropicrin ratio ranging from 20 to 60%, preferably 50%.

17. Extracts as claimed in claim 15 formulated in oils rich in ω-3 and ω-6 polyunsaturated acids.

18. Extracts as claimed in claim 17 formulated in Enothera biennis oil.

19. Extracts as claimed in claim 15 formulated in flax oil.

20. A composition containing the extracts of claim 15.

21. The composition as claimed in claim 20, in the form of tablets, sugar-coated pills, soft- and hard- gelatin capsules and cellulose capsules.

22. The use of the extracts of claim 15 for the preparation of medicaments for the treatment of dyslipidemias, arteriosclerosis and inflammatory bowel disorders.

23. The use of the extracts as claimed in claim 22 for the treatment of irritable colon syndrome.
